(19) United States
(12) Patent Application Publication
(10) Pub. No.: US 2003/0212384 A1
(43) Pub. Date: Nov. 13, 2003

(54) Expandable interventional system

(76) Inventor: Scott William Hayden, Menomonre, WI (US)

Correspondence Address: Scott Hayden, 1720 4th Ave. North, Menomonre, WI, 54751, US

(21) Appl. No.: 10/142,437

(22) Filed: May 10, 2002

(51) Int. Cl. A61M 25/16

(52) U.S. Cl. 604/533

(57) ABSTRACT

The invention provides devices and methods for treatment of acute ischemic stroke and other blockages in small and highly curved or highly diseased vessels and a means of delivering devices and agents to a target site. The devices of the present invention are in a collapsed position while maneuvering to the target lesion that enables access to small vessels. It expands to create a large lumen fill length of the catheter lumen to allow greater suction force and energy than systems known in prior art or a large lumen for easier and safer delivery of an interventional device to a target lesion. The expansion of the distal end also allows the device to seal the vessel at the target site to control flow to the site of a blockage, such as drugs to reduce reprofusion deficit or thrombolytic agents.

### CROSS REFERENCE TO RELATED APPLICATIONS

U.S. Pat, No. 4,646,736

U.S. Pat. No. 5,078,722

U.S. Pat. No. 5,092,839

U.S. Pat. No. 5,908,435

U.S. Pat. No. 5,192,268

U.S. Pat. No. 5,192,291

U.S. Pat. No. 5,282,484

U.S. Pat. No. 5,284,486

U.S. Pat. No. 5,342,297

U.S. Pat. No. 5,356,430

U.S. Pat. No. 5,370,653

U.S. Pat. No. 5,417,703

U.S. Pat. No. 5,419,774

U.S. Pat. No. 5,423,799

U.S. Pat. No. 5,433,495

U.S. Pat. No. 5,480,433

U.S. Pat. No. 5,549,551

U.S. Pat. No. 5,569,275

U.S. Pat. No. 5,578,009

U.S. Pat. No. 5,643,298

U.S. Pat. No. 5,695,507

U.S. Pat. No. 5,730,717

U.S. Pat. No. 5,735,897

U.S. Pat. No. 5,772,674

U.S. Pat. No. 5,800,457

U.S. Pat. No. 5,827,229

U.S. Pat. No. 5,846,251

U.S. Pat. No. 5,851,208

U.S. Pat. No. 5,893,869

U.S. Pat. No. 5,895,400

U.S. Pat. No. 5,902,263

U.S. Pat. No. 5,908,435

U.S. Pat. No. 5,928,218

U.S. Pat. No. 5,954,737

U.S. Pat. No. 5,968,064

U.S. Pat. No. 5,971,938

U.S. Pat. No. 6,001,112

U.S. Pat. No. 6,010,449

U.S. Pat. No. 6,030,397

U.S. Pat. No. 6,090,072

U.S. Pat. No. 6,090,118

U.S. Pat. No. 6,096,054

U.S. Pat. No. 6,120,516

U.S. Pat. No. 6,159,230

U.S. Pat. No. 6,183,443

U.S. Pat. No. 6,206,898

U.S. Pat. No. 6,210,370

U.S. Pat. No. 6,217,549

U.S. Pat. No. 6,238,405

U.S. Pat. No. 6,254,571

U.S. Pat. No. 6,264,671

U.S. Pat. No. 6,270,477

U.S. Pat. No. 6,290,710

U.S. Pat. No. 6,312,444

U.S. Pat. No. 6,312,407

U.S. Pat. No. 6,309,399

U.S. Pat. No. 6,332,886

U.S. Pat. No. 6,364,867

This patent is a continuation of provisional patent application 60/289,653 filed May 10, 2001.

### FIELD OF THE INVENTION

The present invention generally relates to medical devices useful in treating patients with acute stroke or other form of occlusive vascular disease. More specifically, the invention provides a system to create suction to remove a thrombus or embolus lodged in a blood vessel and a means of supplying agents, such thrombolytic or neuroprotective, to the target site for the purpose of reestablishing vascular perfusion.

### BACKGROUND OF THE INVENTION

Stroke is the third most common cause of death in the United States and the leading cause of adult disability. Ischemic strokes are often caused by blood clots or emboli that have dislodged from other body sites or from the cerebral vessels themselves to occlude in the more narrow cerebral arteries downstream.

When treating an ischemic stroke, identifying and opening the occluded vessel during a limited-time window is a significant challenge. Current treatment options have drawbacks. Available therapies include highly invasive craniotomy (remove skull) procedures and slow-acting drug therapy, such as Tissue Plasminogen Activator (tPA). The National Institute of Neurological Disorders and Stroke (NINDS) Tissue Plasminogen Activator for Acute Ischemic Stroke study revealed a 30% reduction in severe disability when tPA was administered within a three-hour window. However, bleeding completions are commonly associated with the use of tPA. Safer and faster-acting interventional therapies, such as mechanical clot removal, could dramatically reduce the extent of the damage. A device that can pass the through the small and highly curved neuro vessels and clear these clots could shift the paradigm of stroke treatment.

After clearing the blockage, the physician can face the challenge of reperfusion deficit (RPD). RPD is the inability to adequately oxygenate brain tissue following the restoration of blood flow. If the patient has RPD, he could continue to suffer the stroke's destructive effects even after the blockage has been removed. Drugs, such as nicardipine, are showing potential for lessening RPD. Therefore, a device should also allow control of reperfusion back into the vessels.

A device or system that can quickly open occluded vessels offers the potential to improve patients' lives dramatically. The ischemic stroke treatment area is very exciting, but also the most challenging. It is exciting because treating strokes and rapidly restoring blood flow offers the potential to improve patient health dramatically and immediately. The challenge of current mechanical clot removal systems, however, is reaching safely and quickly. The clot removal system of the invention is small and highly maneuverable, allowing access to small and highly curved vessels located deep within the brain.

Many systems and catheters are known in prior art that create suction to remove unwanted material from vessels, including a system using retrograde flow to create a vacuum at the distal end of the catheter, rotating systems using a helical pattern with metal blades or burrs, and catheters that expand at the distal tip or end but not the full length of the lumen. Many of these systems are either large or stiff to reach many target sites, or cannot produce sufficient suction force, energy, or power to remove the blockage.

For other interventional procedures, large lumen delivery systems are also desired for delivery of interventional devices, such as vascular stents, angioplasty balloons and the like. A catheter with a large lumen extending the full length of the catheter body would allow easier passage of these devices to a target site. A system that provides a conduit from the insertion site to the target site can protect the vessels against unwanted complications, such as perforating a vessel or loosing a device in the vessel.

### SUMMARY OF THE INVENTION

The invention provides devices and methods for treatment of acute ischemic stroke and other blockages in small and highly curved or highly diseased vessels and a means of delivering devices and agents to a target site. The devices of the present invention are in a collapsed position while maneuvering to the target lesion that enables access to small vessels. It expands to create a large lumen for the full length of the catheter lumen to allow greater suction force and energy than systems known in prior art and/or a large lumen for easier and safer delivery of interventional devices to a target lesion. The expansion of the distal end also allows the device to seal the vessel at the target site to control flow at the site of a blockage, such as drugs to reduce reperfusion deficit or thrombolytic agents.

A first embodiment of the medical device comprises a collapsible/expandable catheter and impeller, and an external suction source. The catheter has a noncompliant proximal section, a collapsible/expandable distal section and a lumen that communicates with an aspiration port at the distal end. The distal section may comprise of an tube inflatable bladders or reservoirs in walls, which communicates with an inflation lumen at the proximal portion of the catheter to expanded and collapse, or of at least one layer highly lubricous tubing that can expanded radially by the movement of the rotating impeller mounted on a flexible drive shaft. Reducing the profile by collapsing allows the system access to small or heavily diseased vessels. The impeller may be operated to produce suction, expand the outer sheath, and/or produce pulling of the system via fluid movement to assist in reaching the target location. Rotation of the impeller is controlled by an external drive mechanism. An external pump, such as a manual piston pump, may be connected to the proximal port communicating with the aspiration port to produce suction.

In another embodiment, the catheter has a noncompliant proximal section, a collapsible/expandable distal section and a lumen that communicates with an aspiration port at the distal end. The distal section may comprise of an tube inflatable bladders or chambers in walls, which communicates with an inflation lumen to expanded and collapse, or of at least one layer highly lubricous tubing that can expanded radially by a separate dilatation system, such as a angioplasty-type balloon. An external pump, such as a piston-type displacement pump, can create in the lumen either pressure to assist in the delivery of an interventional device through the lumen of the system, or to create suction to remove unwanted material from the vessel. In a variation of this embodiment, a piston-type displacement pump may also be created near the distal end of the expandable catheter by forming a piston and cylinder by expanding a piston-type object, such as with a balloon, in the catheter body near the tip and then pulled back toward the proximal port to create the suction needed to draw the blockage into the body of the catheter for subsequent remove from the artery.

### BRIEF DESCRIPTION OF THE SEVERAL VIEWS OF THE DRAWINGS

FIG. 1 is a side view of distal section in the collapsed state illustrating position within an occluded blood vessel.

FIG. 2 is a side view of distal section in the expanded state illustrating position within an occluded blood vessel.

FIG. 3 is a side view of inflatable system in the collapsed state.

FIG. 4 is a side view of inflatable system in the expanded state.

FIG. 5 is a cross sectional side view of the inflatable distal section in the expanded state.

FIG. 6 is a cross sectional view of inflatable distal section in the expanded state.

FIG. 7 is another cross sectional view of inflatable distal section in the expanded state.

FIG. 8 is a side view of distal pump system in the collapsed state.

FIG. 9 is a cross sectional side view of distal pump configuration of the piston pump in the collapsed state illustrating position within an occluded blood vessel.

FIG. 10 is a cross sectional side view of distal pump configuration of the piston pump in the expanded state illustrating position within an occluded blood vessel.

FIG. 11 is a cross sectional side view of collapsible impeller system in the collapsed state illustrating position within an occluded blood vessel.

FIG. 12 is a cross sectional side view of collapsible impeller system in the collapsed state illustrating position within an occluded blood vessel.

FIG. 13 is a side view of collapsible impeller system in the collapsed state.

### DETAILED DESCRIPTION OF THE DRAWINGS

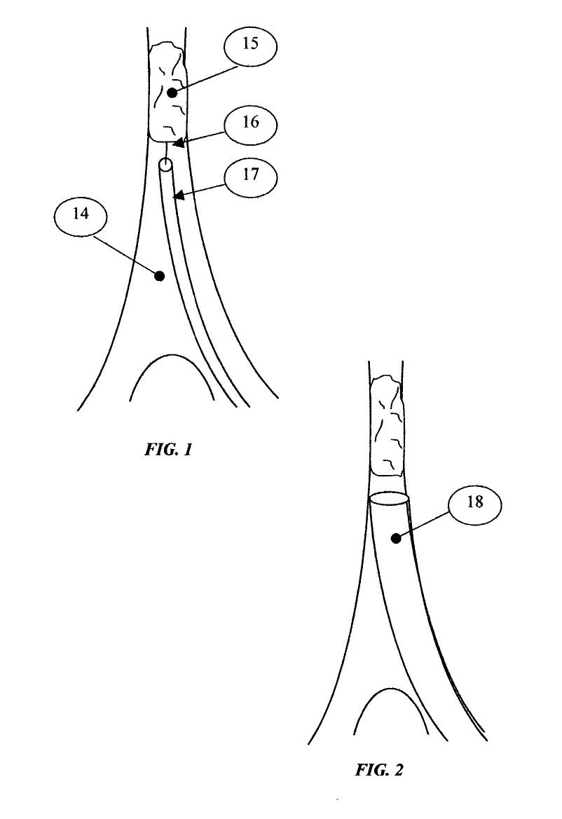

Referring to FIGS. 1 and 2, an interventional system that is an embodiment of the present invention is shown. It is to be understood that embodiments of the invention may take the form of a typical delivery catheter or system, aspiration system or the like.

FIGS. 1 and 2 show the catheter being used in accordance with the method of the invention to remove an occlusion from a vessel. It is to be understood that use of the device in a blood vessel to remove blockage is presented as an example only and that the system and method of the present invention may be used to remove a variety of undesirable materials from a number of different tubular structures in the human body. The latter includes, but is not limited to, tubular structures of the biliary, excretory and vascular systems.

As shown in FIGS. 1 and 2, a guide wire 16 has been inserted into the blood vessel 14 near an occlusion 15. Next, catheter body 17 is introduced into blood vessel 1 with or without an expandable impeller or piston disposed in the distal section of the catheter. The blunt end of the catheter body 17 does not damage the walls of vessel 14 as it is advanced. After reaching the target site, distal end 17 is expanded. Once the catheter body is expanded 18, as shown in FIG. 2, suction is applied through distal tip, pulling blockage material into lumen of catheter body. At this point, the system may be collapsed and /or withdrawn if desired. However, it may also be desirable to inject an agent, such as tPA, urokinase, nicarpodine or the like before the restoring physiologic blood flow and removing the system.

The diameter of the guidewire 16 is generally in the range of about 0.07 inches to about 0.014 inches. The length of the guidewire 16 may be varied to correspond to the distance between the percutaneous access site and the lesion being operated upon. In an application for removing blockage from a cerebral or coronary artery by way of a femoral artery access, guidewires 16 having lengths from about 180 cm to about 300 cm may be used as will be understood by those of skill in art.

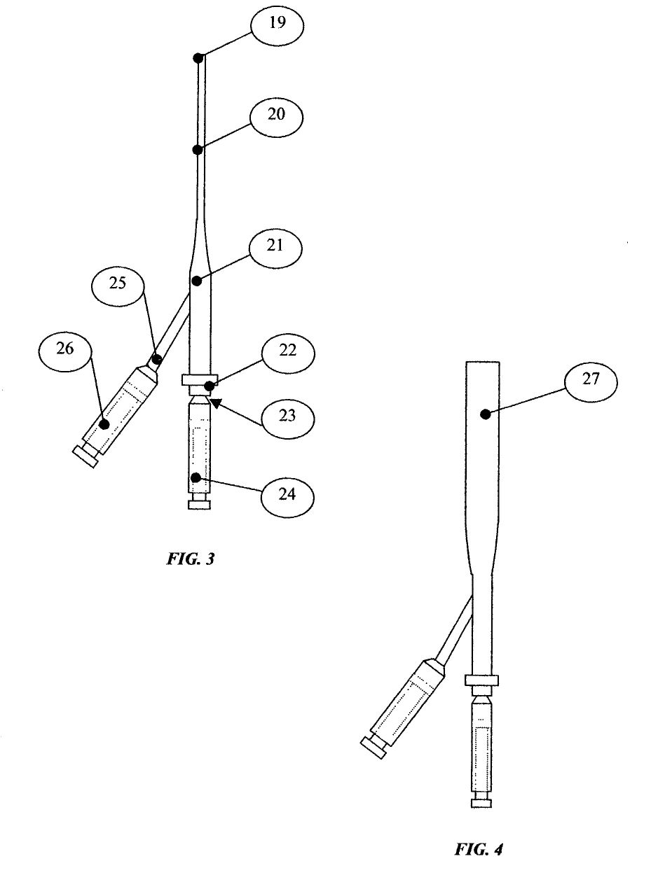

In FIG. 3, distal catheter body 20 is shown with the distal end of the lumen opening 19 in the collapsed state. The diameter of expanded opening 19 is chosen depending upon the size of the tubular structure of the human body within which the catheter is placed. Opening 19 maybe expanded 27 as shown in FIG. 4 with the intent of engaging the interior of the wall of the tubular structure so as to create a circumferential occlusive seal therein. However, in situations in which flow through the tubular structure cannot be completely interrupted, as, for example, in a main artery, the expanded diameter may be chosen so as to create an enlarged orifice, but without circumferential contact with the interior of the wall of the tubular structure. Flow may then continue around distal opening 19.

The catheter cylindrical proximal body 21 features Luer Lock hub 22 mounted on its proximal end and a central lumen 23 through which a guide wire and other devices may be passed. The proximal portion 21 of catheter body diameter is fixed so as to provide rigidity. Proximal catheter body 21 is preferably constructed of at least one plastic polymer and/or a metallic substance. A common configuration is polyurethane, polyester or polyamide base outer layer, lubricous inner layer and a braided or wound stainless steel structure imbedded between the two polymers. The distal portion of catheter body 20 may at least one polymer, such as elastomeric material. The distal portion 20 may be collapsed radially by folding a non-compliant material or by stretching a compliant material, such as polyurethane or amide base polymers.

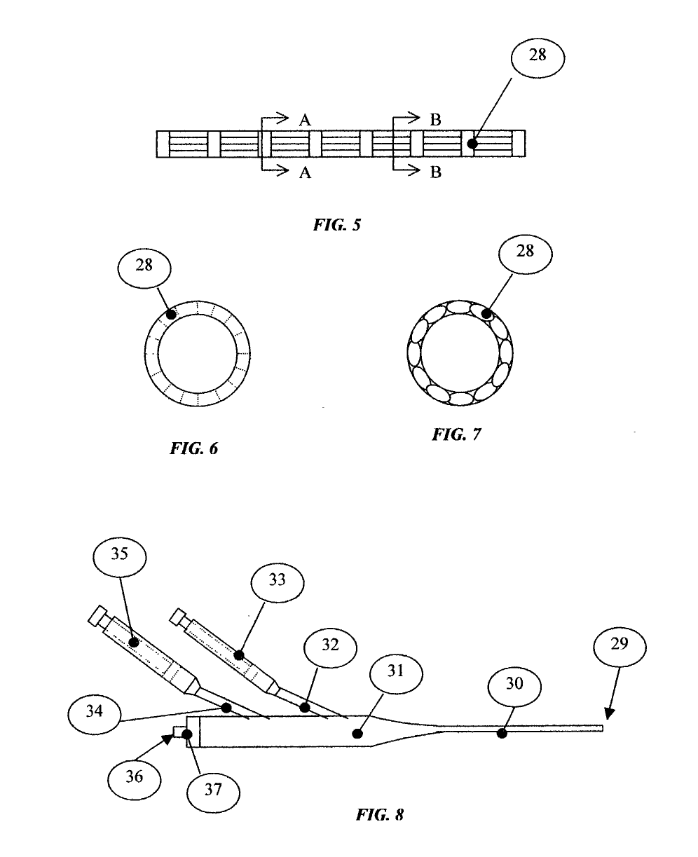

The distal section of the catheter may also be expanded by inflation of bladders or reservoirs comprising the wall as shown in FIGS. 5-7. The section is inflated so that the distal end 19 of distal catheter body 20 is able to accommodate occlusion material. The section is inflated in the same manner as a balloon catheter through side port 25 with injection system 26. Such balloon catheters are well known in the art. Once expanded suction can be applied through the proximal lumen port 23 by suction pump 24 to remove the blockage.

The inflatable distal section 28 is designed to minimize wall thickness while maintaining adequate radial support in the expanded state. It may be comprised of many different patterns formed by bonding two or more using biologically inert adhesives or thermobonding or with multi-lumen extruded tubing. The inflatable distal section is preferably composed of a slightly elastic plastic polymer that is biologically inert and expands to a predictable degree under inflation pressure. Plastics such as polyamide or polyurethane and the like may be used for this purpose.

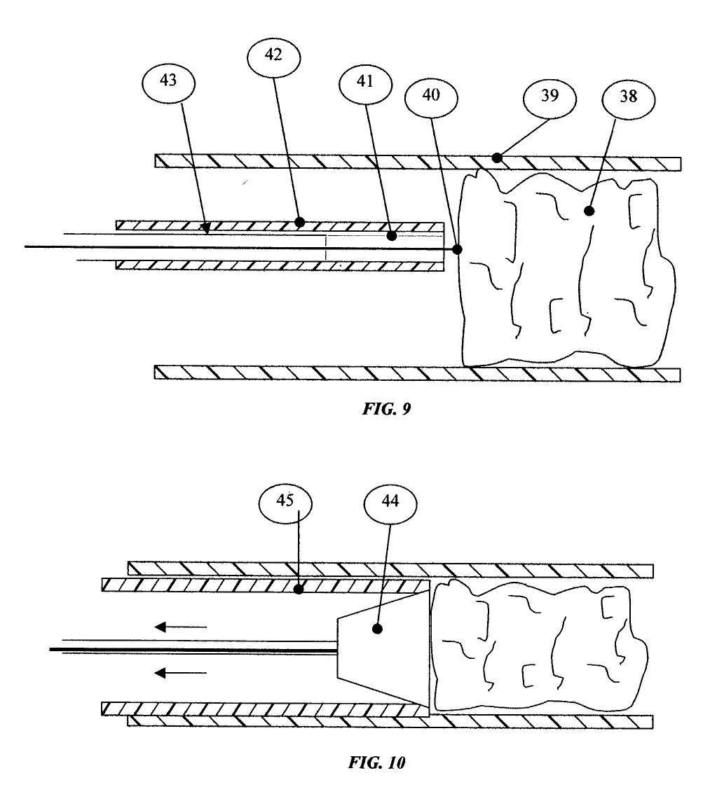

FIGS. 8-10 show another embodiment where an expandable piston 41 is expanded 44 in the distal section 30 to increase lumen size of the distal catheter 30 and possibly engage the vessel wall 39 to form a piston-type displacement pump with the wall of the expanded catheter 45. The system is in a collapsed position while tracking over a guidewire 40 to access the location of the blockage 38. The expandable piston may comprise of a balloon 41 inflated by an injection system 35 through a side port 34 communicating with an inflation lumen 43. While a balloon type piston is used in this example, an alternative design with a non-inflatable system using a may be used. In this case, side port 34 and pump 35 on FIG. 8 may be eliminated. Note the proximal portion of the proximal shaft 31, including the side port 34, pump 35, proximal hub 37, may be separated from the distal portion of the proximal body of the catheter to allow pull back of the piston from the main body of the catheter.

In FIG. 8, distal catheter body 30 is shown with the distal end of the lumen opening 29 in the collapsed state. Opening 29 maybe expanded as shown in FIG. 10 with the intent of engaging the interior of the wall of the tubular structure so as to create a circumferential occlusive seal therein. The catheter cylindrical proximal body 31 features Luer Lock hub 37 mounted on its proximal end and a central lumen 36 through which a guide wire and other devices may be passed. The proximal portion 31 of catheter body diameter is fixed so as to provide rigidity. The distal portion 30 may be collapsed radially by folding a non-compliant material or by stretching a compliant material, such as polyurethane or amide base polymers. Once the system is expanded, pull back of the system is achieved by pulling the guidewire and the proximal portion of the catheter to product suction to draw the blockage into the distal end of the catheter body 30 through the aspiration port 29.

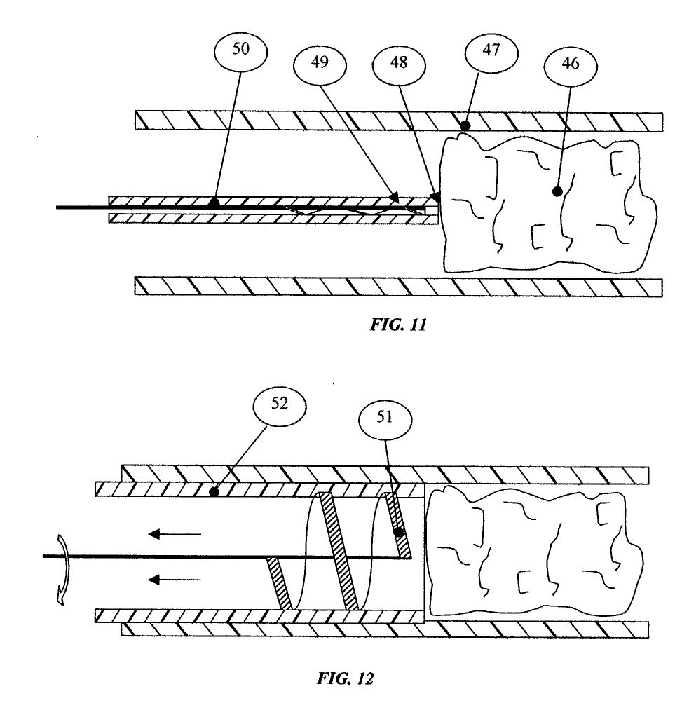

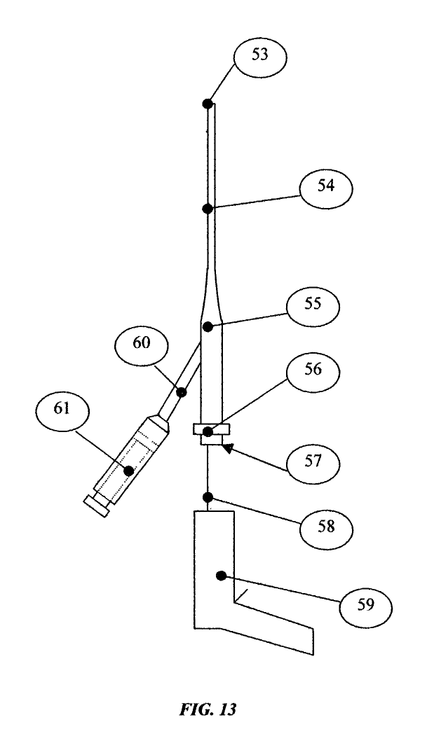

In another embodiment FIGS. 11-13, an illustrative embodiment featuring a collapsible impeller is shown. In general, the device comprises a collapsible distal catheter body 54, fixed diameter proximal body 55, with a communicating lumen from having a proximal port 57 to the distal port 53. A drive shaft control 59 is provided on the proximal end of the tubular body for permitting manipulation of the impeller 49, 51 in the distal end. The proximal port features Luer Lock hub 56 for attachment of connectors. A side port 60 allows communication with the catheter lumen to allow suction or injection of fluid via a pump 61.

Referring to FIGS. 11 and 12, the catheter distal section 52 is provided with an aspiration port 48 and an collapsible flexible impeller 49. The drive shaft 22 is rotationally coupled to the control 59 by way of an elongate flexible drive shaft 50. The drive shaft 50 is rotated by the drive shaft drive control 59 to expand the impeller 51 and the distal section of the catheter 52 to produce suction the, if desired, to form an occlusive seal with the artery.

The flexible, collapsible helical impeller can be formed of an injection molded or machined elastomeric polymer material, or constructed with a coiled wire of superelastic material, such as nickel titanium, covered with a elastomeric polymer.

Although this invention has been described in terms of certain preferred embodiments, other embodiments apparent to those of ordinary skill in the art are also within the scope of this invention. Accordingly, the scope of this invention is intended to be defined only by the claims that follow.

1. A medical device for removing blockage in an artery comprised of;
an enlongated catheter having a proximal section, a distal section, and a lumen there between communicating with an aspiration port at the distal end with an radially expandable distal section that is greater than 1 centimeter long and a non-compliant proximal section; and
a piston-type pump to create suction or pressure in catheter lumen.

2. The device of claim 1, wherein said distal section of catheter is inflatable causing the distal section to increase lumen size.

3. The device of claim 1, wherein the distal section of catheter is comprises of at least one polymer that is expanded or unfolded upon expansion.

4. The device of claim 3, wherein a metal component is embedded between polymer.

5. The device of claim 1, wherein the proximal end of the catheter is adapted for attachment to a source for suction.

6. The device of claim 1, wherein the aspiration port and lumen are adapted for infusion of fluid and pharmaceutical agents.

7. The device of claim 1, wherein the piston-type pump is attached to proximal port.

8. The device of claim 1, wherein the piston-type pump consist of a expandable member in distal end of expandable catheter.

9. A medical device for removing blockage in an artery comprised of;
an enlongated catheter having a proximal end, a distal end, and a lumen there between communicating with an aspiration port at the distal end with an radially expandable distal section that is greater than 1 centimeter long and a non-compliant proximal section;
a rotatable element extending through the body;
a collapsible rotatable tip at the distal end of the body and connected to the rotatable element;
an annular space between the rotatable tip and an interior wall of the tubular body; and
a drive means for rapidly rotating the shaft.

9. The device of claim 9, wherein said distal section of catheter is inflatable causing the distal section to increase lumen size.

10. The device of claim 9, wherein the distal section of catheter is comprises of at least one polymer tube that is unfolded or stretched upon expansion.

11. The device of claim 9, wherein the proximal end of the catheter is adapted for attachment to a source for suction.

12. The device of claim 9, wherein the aspiration port and lumen are adapted for infusion of fluid and pharmaceutical agents.

13. The device of claim 9, wherein the rotatable tip is comprised of a helical metal wire and polymer forming the blade.

14. The device of claim 9, wherein the rotatable tip is comprised of at least one polymer to form the blade.

15. A method for removing blockage from an artery, comprising the steps of:
positioning a system comprised of a rotatable shaft with collapsible impeller on distal portion and an enlongated catheter having a proximal end, a distal end, and a lumen there between communicating with an aspiration port at the distal end with an radially expandable distal section and a non-compliant proximal section;
expanding the distal section to in increase lumen size;
rotating the impeller at a speed greater than 60 revolutions per minute;
applying a negative pressure to the aspiration port, wherein the blockage material is engaged by the port;

16. A method as in claim 15, wherein an injection of a thrombolytic or reperfusion deficient agent may be administered.

17. A method as in claim 15, wherein the expanded the enlongated catheter distal section is collapsed before device removal from body.

18. A method for removing thromboembolic material from an artery, comprising the steps of:
positioning a guidewire near the target site;
tracking over the wire to the target site a catheter having a proximal section, a expandable distal section, and a lumen there between communicating with an aspiration port at the distal end;
inserting the distal end of the catheter into the artery;
expanding the distal section to increase lumen size of distal section;
applying a negative pressure to the aspiration port, wherein the thromboembolic material is engaged by the port.

19. A method as in claim 18, wherein an injection of a thrombolytic or reperfusion deficient agent may be administered.

20. A method as in claim 18, wherein the expanded the enlongated catheter distal section is collapsed before device removal from body.
